(19) United States
(12) Patent Application Publication
(10) Pub. No.: US 2013/0104300 A1
(43) Pub. Date: May 2, 2013

(54) DEVICE FOR DETACHABLY FIXING LENS ON GOGGLES

(71) Applicant: KOREA OGK CO., LTD., Wonju-si (KR)

(72) Inventor: Soo An PARK, Wonju-si (KR)

(73) Assignee: KOREA OGK CO., LTD., Wonju-si (KR)

(21) Appl. No.: 13/665,286

(22) Filed: Oct. 31, 2012

(30) Foreign Application Priority Data
Oct. 31, 2011 (KR) 10-2011-0009652

(51) Int. Cl. A61F 9/02

(52) U.S. Cl. 2/439

(57) ABSTRACT

The present invention relates to a device for detachably fixing a lens on goggles including: a detachable fixing member having a plurality of metal pieces adapted to be insertedly fixed to a plurality of equally spaced insertion grooves formed along a frame rim and a plurality of permanent magnets adapted to be insertedly fixed to a plurality of equally spaced insertion grooves formed along a lens rim on the same line as the plurality of metal pieces insertedly fixed along the frame rim in such a manner as to be coupled and separated to and from the plurality of metal pieces; and clip members adapted to be fixed to both sides of the frame so as to rigidly fix the lens to the frame.

### BACKGROUND OF THE INVENTION

1. Field of the Invention

The present invention relates to a device for detachably fixing a lens on goggles, and more particularly, to a device for detachably fixing a lens on goggles wherein the lens, which is adapted to be coupled to the front surface of a frame of the goggles to provide a clear front view to a wearer, is firmly fixed doubly to the frame by means of magnets and clip members and is also separated easily from the frame if necessary.

2. Background of the Related Art

Goggles are worn to protect a wearer's eyes during various activities like ski, sky diving, paragliding, and so on and largely include a frame with bands attached at both sides thereof and a lens coupled to the front surface of the frame.

Means for fixing the lens to the frame includes a plurality of equally spaced hooks mounted on the back surface of a lens rim and fastening grooves formed on the inner surface of the frame in such a manner as to insert the hooks thereinto, such that the frame and lens are assembled as a unitary body with each other.

If the existing lens is desired to change new one, under the above-mentioned structure, the lens cannot be separated from the frame, and in the conventional practice, therefore, it is very hard to change the lens on the goggles to other lenses.

So as to remove the above-mentioned problems, thus, there has been proposed Korean Utility Model Registration No. 20-2010-12001 filed by the same applicant as the invention, wherein goggles have a structure wherein a lens is easily fixed to a frame.

In the prior art, however, the lens can be rapidly and easily fixed to the frame by means of clips, but once fixed, the lens cannot be separated from the frame, which of course makes impossible to change the lens to new one. It can be therefore understood that the prior art has a disadvantage that basically makes the goggles not used with various colored lenses.

### SUMMARY OF THE INVENTION

Accordingly, the present invention has been made in view of the above-mentioned problems occurring in the prior art, and it is an object of the present invention to provide a device for detachably fixing a lens on goggles wherein various colored lenses can be detachably fixed to the front surface of a frame of the goggles in rapid and easy manners.

It is another object of the present invention to provide a device for detachably fixing a lens on goggles wherein the lens is fixed doubly to a frame by means of a magnet member and clip members, such that the fixed lens and frame are not separated from each other even at the application of substantially strong impacts to the goggles.

To accomplish the above objects, according to the present invention, there is provided a device for detachably fixing a lens on goggles, wherein the goggles have a frame having sponge disposed on the inner surface thereof and a frame rim disposed on the outer surface thereof and the lens having a lens rim disposed on the back surface thereof in such a manner as to be coupled to the front surface of the frame, the device including: a detachable fixing member having a plurality of metal pieces adapted to be insertedly fixed to a plurality of equally spaced insertion grooves formed along the frame rim and a plurality of permanent magnets adapted to be insertedly fixed to a plurality of equally spaced insertion grooves formed along the lens rim on the same line as the plurality of metal pieces insertedly fixed along the frame rim in such a manner as to be coupled and separated to and from the plurality of metal pieces; and clip members adapted to be fixed to both sides of the frame so as to rigidly fix the lens to the frame.

According to the present invention, preferably, each of the clip members includes: a fixing piece having fastening protrusions formed on the upper and lower portions thereof in such a manner as to be insertedly fixed to both sides of the frame, a band-coupling hole extended vertically on the outer surface thereof in such a manner as to be insertingly coupled to the inner end of a band, a shaft insertion hole formed on the center of the front surface thereof in such a manner as to be insertingly coupled to a shaft disposed on the inner surface of a lever, and a guide slot formed circumferentially on the outside of the shaft insertion hole in such a manner as to insert a guide protrusion formed on the inner surface of the lever thereinto; the lever having the shaft and the guide protrusion formed on the inner surface thereof, a support protrusion formed on the outer surface thereof in such a manner as to be coupled to a slide slot formed on the cover, and a hook formed on the inner end thereof in such a manner as to be coupled and separated to and from a hook mounting piece formed on both sides of the lens rim; and the cover having fastening grooves formed on the upper and lower portions of the inner surface thereof in such a manner as to be coupled to the fastening protrusions formed on the fixing piece and the slide slot formed on the center portion thereof in such a manner as to be insertingly coupled to the support protrusion formed on the outer surface of the lever.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a perspective view showing a configuration of a frame rim in a device for detachably fixing a lens on goggles according to the present invention;

FIG. 2 is a perspective view showing a configuration of a lens rim in the device for detachably fixing a lens on goggles according to the present invention;

FIGS. 3 and 4 are exploded perspective views showing a clip member in the device for detachably fixing a lens on goggles according to the present invention;

FIG. 5 is a perspective view showing a state wherein the clip member is coupled to the frame in the device for detachably fixing a lens on goggles according to the present invention;

FIG. 6 is a perspective view showing a state wherein the lens is coupled to the frame in the device for detachably fixing a lens on goggles according to the present invention;

FIG. 7 is a perspective view showing the assembling state of the device for detachably fixing a lens on goggles according to the present invention; and

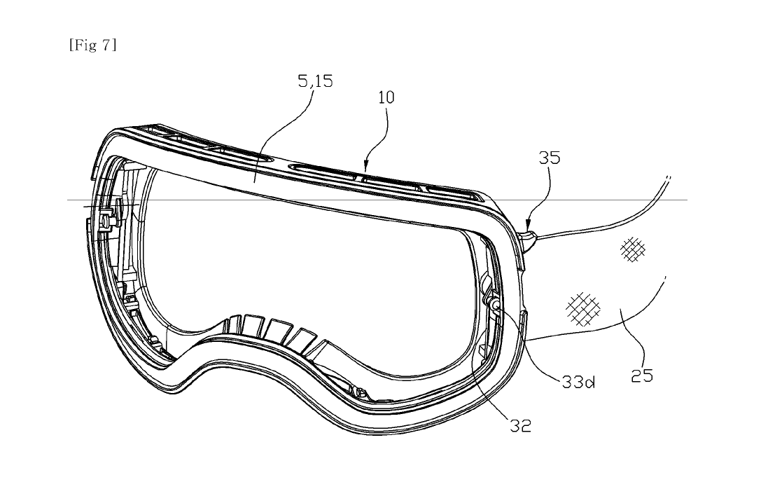

FIG. 8 is a perspective view showing the disassembling state of the device for detachably fixing a lens on goggles according to the present invention.

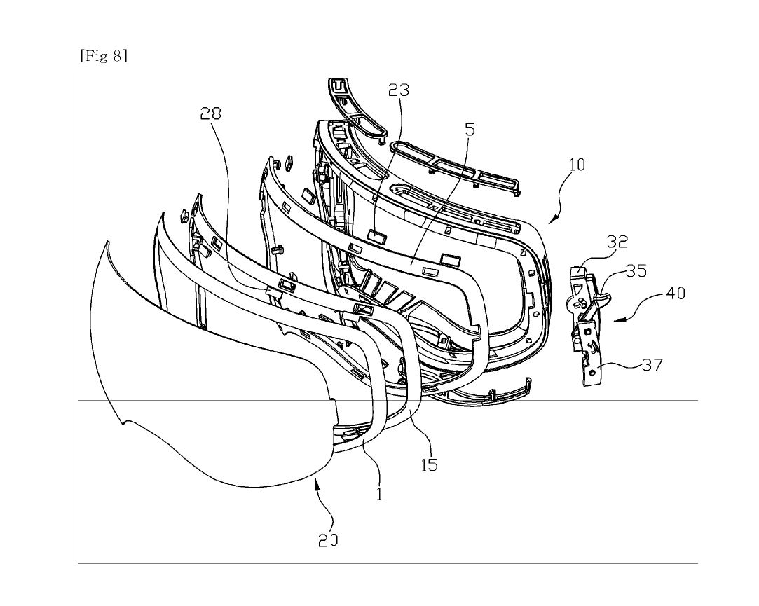

### DETAILED DESCRIPTION OF THE PREFERRED EMBODIMENT

Hereinafter, an explanation on a device for detachably fixing a lens on goggles according to the present invention will be in detail given with reference to the attached drawing.

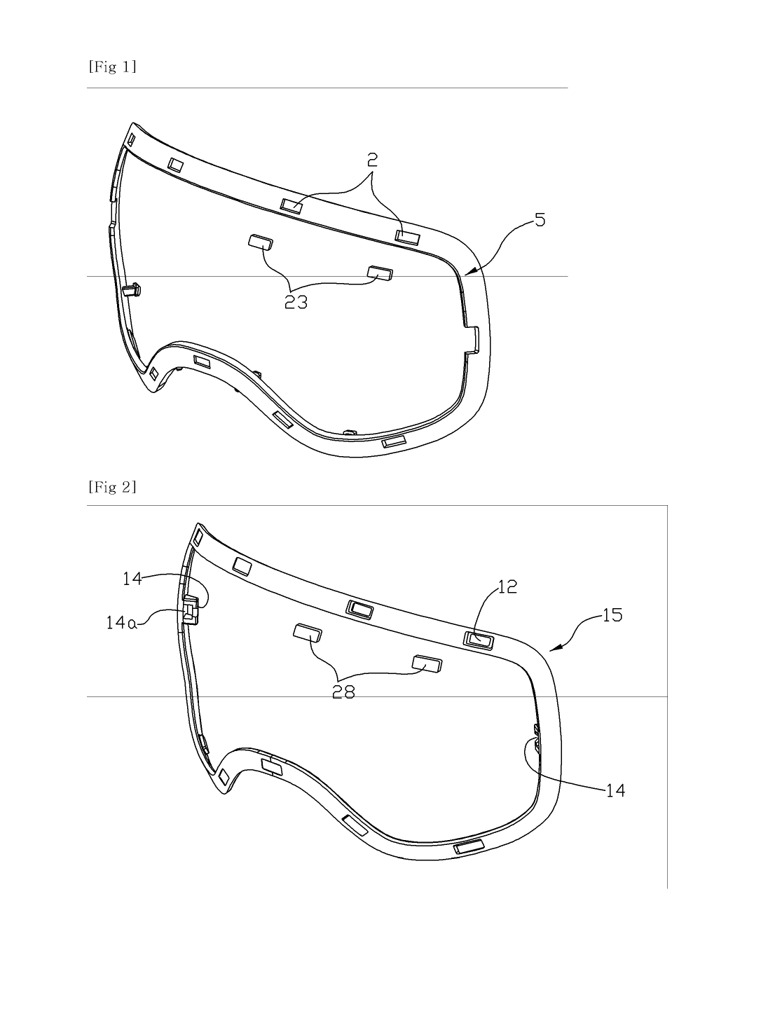

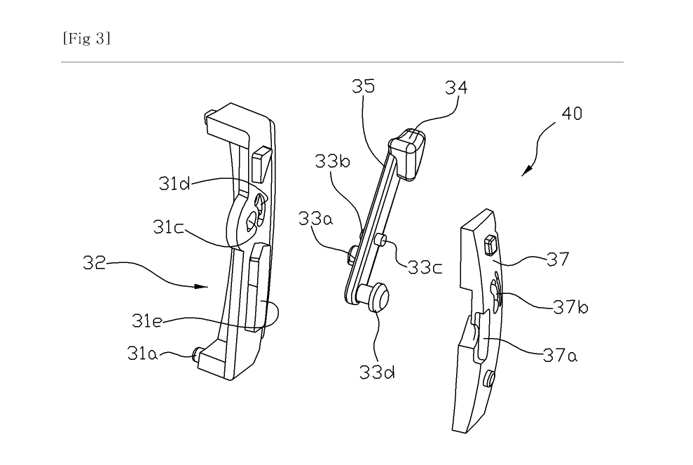

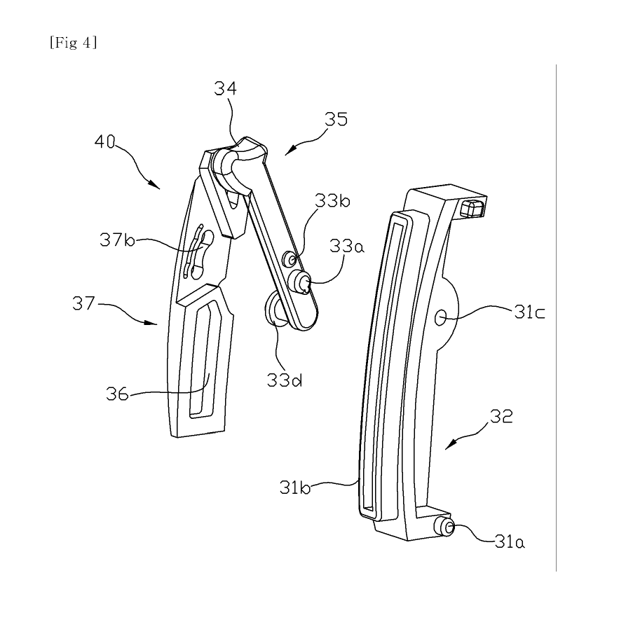

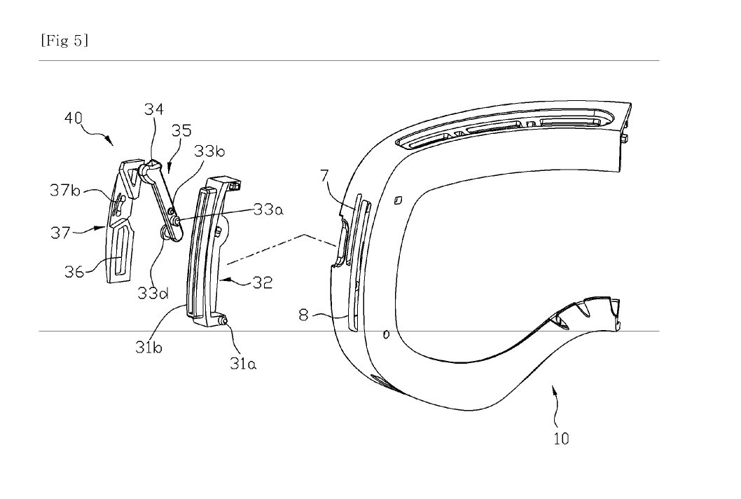

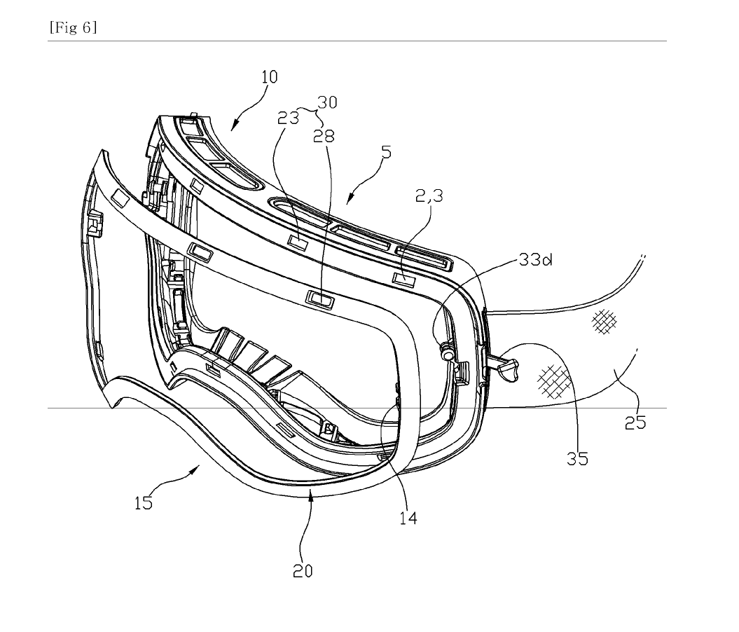

FIG. 1 is a perspective view showing a configuration of a frame rim in a device for detachably fixing a lens on goggles according to the present invention, FIG. 2 is a perspective view showing a configuration of a lens rim in the device for detachably fixing a lens on goggles according to the present invention, FIGS. 3 and 4 are exploded perspective views showing a clip member in the device for detachably fixing a lens on goggles according to the present invention, FIG. 5 is a perspective view showing a state wherein the clip member is coupled to the frame in the device for detachably fixing a lens on goggles according to the present invention, and FIG. 6 is a perspective view showing a state wherein the lens is coupled to the frame in the device for detachably fixing a lens on goggles according to the present invention.

As shown, the present invention relates to a device for detachably fixing a lens on goggles wherein a lens 20 adapted to be coupled integrally to the front surface of a frame 10 is coupled and separated to and from the frame 10 in easy and rapid manners, and once coupled, further, the lens 20 is rigidly fixed to the frame 10.

The device for detachably fixing a lens on goggles according to the present invention largely includes a detachable fixing member 30 adapted to detachably fix the lens 20 to the front surface of the frame 10 and clip members 40 adapted to rigidly fix the lens 20 to the frame 10 after the lens 20 has been coupled to the frame 10.

The detachable fixing member 30 includes a plurality of metal pieces 23 adapted to be insertedly fixed to a plurality of equally spaced insertion grooves 2 formed along a frame rim 5 bonded integrally to the front surface of the frame 10 constituting the goggles and a plurality of permanent magnets 28 adapted to be insertedly fixed to a plurality of equally spaced insertion grooves 12 formed along a lens rim 5 bonded integrally to the back surface of the lens 20 by means of a hot-melt tape 1 applied to the back surface of the lens 20.

The plurality of equally spaced metal pieces 23 and permanent magnets 28 along the frame rim 5 and the lens rim 15 are positioned on the same line as each other, such that the lens 20 can be coupled and separated to and from the frame 10 through the magnetic forces of the permanent magnets 28.

On the other hand, the clip members 40, which are disposed on both sides of the frame 10 so as to rigidly fix the lens 20 to the frame 10, each have the configuration as shown in FIGS. 3 and 4.

Each of the clip members 40 has a fixing piece 32 adapted to be fixed to each side of the frame 10, a lever 35 adapted to be coupled to the fixing piece 32 in such a manner as to be moved upwardly and downwardly, and a cover 37 adapted to be coupled integrally to the lever 35 on the outside thereof.

The fixing piece 32 has fastening protrusions 31a formed on the upper and lower portions thereof in such a manner as to be insertedly fixed to fastening holes 3 formed on the frame 10 and a band-coupling hole 31b extended vertically on one side thereof in such a manner as to be insertedly fixed to an elongated hole 8 formed on one side of the frame 10, the band-coupling hole 31b being coupled to the inner end of a band 15.

Further, the fixing piece 32 has a shaft insertion hole 31c formed on the center of the front surface thereof in such a manner as to be insertingly coupled to a shaft 33a of the lever 35 and a guide slot 31d formed circumferentially on the outside of the shaft insertion hole 31c so as to guide the stable movement of the lever 35.

On the other hand, the fixing piece 32 further includes fastening protrusions 31e protruded outwardly on the upper and lower sides of the shaft insertion hole 31c in such a manner as to be coupled to the cover 37 as will be discussed later.

The lever 35, which is operated upwardly and downwardly, includes the shaft 33a and a guide protrusion 33b formed on the inner surface thereof in such a manner as be insertedly coupled to the shaft insertion hole 31c and the guide slot 31d of the fixing piece 32 and a support protrusion 33c formed integrally on the outer surface thereof in such a manner as to be insertedly coupled to a slide slot 37b formed on the cover 37.

Further, the lever 35 includes a hook 33d formed on the inner end thereof in such a manner as to be coupled and separated to and from a hook mounting piece 14 formed on both sides of the lens 20 as will be discussed later and a handle 34 formed on the outer end thereof in such a manner as to be exposed to the outside of the frame 10.

On the other hand, so as to prevent the lever 35 fastened to the fixing piece 32 from being escaped therefrom and to help the stable operation of the lever 35, the cover 37 has fastening grooves 36 formed on the upper and lower portions of the inner surface thereof in such a manner as to be coupled to the fastening protrusions 31e formed on the fixing piece 32, and in this case, each of the fastening grooves 36 has a generally fan-like shape whose inside becomes wide and whose outside becomes narrow.

Such fan-like shape allows the lever 35 to be operated within a given section.

The cover 37 further has an incised groove 37a formed on one side of the outer surface thereof in such a manner as to insert the hook mounting piece 14 molded integrally to the lens rim 15 and the slide slot 37b formed on the center portion thereof in such a manner as to induce the support protrusion 33c formed on the outer surface of the lever 35.

The hook mounting piece 35 to and from which the hook 33d of the lever 35 is coupled and separated is formed integrally on both sides of the lens rim 15 in such a manner as to be insertedly fixed to the incised groove 37a formed on the outer surface of the cover 37.

So as to couple the lens 20 selected by a user to the front surface of the frame 10, according to the present invention, the permanent magnets 28 fixed to the lens rim 15 bonded integrally to the back surface of the lens 20 are coupled just to the metal pieces 23 insertedly fixed to the frame rim 5, thereby finishing the integral coupling between the frame 10 and the lens 20.

Further, if the levers 35 mounted on both sides of the frame 10 move upwardly, the hooks 33d of the levers 35 move downwardly and are thus inserted to concave grooves 14a formed on the hook mounting pieces 14, thereby achieving the rigid fixing of the lens 20.

Contrarily, if the lens 20 is changed to other lenses, the levers 35 move downwardly, so that the hooks 33d inserted to the concave grooves 14a of the hook mounting pieces 14 are separated therefrom, thereby achieving the separation of the lens 20 from the frame 10.

Like this, if the hooks 33d supporting the lens 20 are separated, the lens 20 coupled to the front surface of the frame 10 is detached, and in the same manner as above, thus, new lens 20 is coupled to the frame 10.

As described above, there is provided the device for detachably fixing a lens on goggles wherein the lens is detachably fixed to the frame by means of the magnet member and clip members, such that various colored lenses can be selected and worn in accordance with a user's preference and wherein the lens are not escaped from the frame even at the application of substantially strong impacts to goggles.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. A device for detachably fixing a lens on goggles, wherein the goggles have a frame having sponge disposed on the inner surface thereof and a frame rim disposed on the outer surface thereof and the lens having a lens rim disposed on the back surface thereof in such a manner as to be coupled to the front surface of the frame, the device comprising:
a detachable fixing member having a plurality of metal pieces adapted to be insertedly fixed to a plurality of equally spaced insertion grooves formed along the frame rim and a plurality of permanent magnets adapted to be insertedly fixed to a plurality of equally spaced insertion grooves formed along the lens rim on the same line as the plurality of metal pieces insertedly fixed along the frame rim in such a manner as to be coupled and separated to and from the plurality of metal pieces; and
clip members adapted to be fixed to both sides of the frame so as to rigidly fix the lens to the frame.

2. The device for detachably fixing a lens on goggles according to claim 1, wherein each of the clip members comprises:
a fixing piece having fastening protrusions formed on the upper and lower portions thereof in such a manner as to be insertedly fixed to both sides of the frame, a band-coupling hole extended vertically on the outer surface thereof in such a manner as to be insertingly coupled to the inner end of a band, a shaft insertion hole formed on the center of the front surface thereof in such a manner as to be insertingly coupled to a shaft disposed on the inner surface of a lever, and a guide slot formed circumferentially on the outside of the shaft insertion hole in such a manner as to insert a guide protrusion formed on the inner surface of the lever thereinto;
the lever having the shaft and the guide protrusion formed on the inner surface thereof, a support protrusion formed on the outer surface thereof in such a manner as to be insertedly coupled to a slide slot formed on the cover, and a hook formed on the inner end thereof in such a manner as to be coupled and separated to and from a hook mounting piece formed on both sides of the lens rim; and
the cover having fastening grooves formed on the upper and lower portions of the inner surface thereof in such a manner as to be coupled to the fastening protrusions formed on the fixing piece and the slide slot formed on the center portion thereof in such a manner as to be insertingly coupled to the support protrusion formed on the outer surface of the lever.
